(19) United States
(12) Patent Application Publication
(10) Pub. No.: US 2019/0329244 A1
(43) Pub. Date: Oct. 31, 2019

(54) SIEVE-THROUGH VERTICAL FLOW SYSTEM FOR PARTICLE-BASED BIOASSAYS

(71) Applicant: AGENCY FOR SCIENCE, TECHNOLOGY AND RESEARCH, Singapore (SG)

(72) Inventors: Jackie Y. YING, Singapore (SG); Yi ZHANG, Singapore (SG); Yoke San LEE, Singapore (SG); Aysha FARWIN, Singapore (SG)

(21) Appl. No.: 16/476,048

(22) PCT Filed: Jan. 4, 2018
(86) PCT No.: PCT/SG2018/050005

§ 371 Date: Jul. 3, 2019

(30) Foreign Application Priority Data
Jan. 4, 2017 (SG) 10201700040Y

(51) Int. Cl. B01L 3/00; G01N 33/543

(52) U.S. Cl. CPC B01L 3/50255; G01N 33/54313; B01L 2400/0406; B01L 2300/0681; B01L 2300/069; B01L 2200/0668

(57) ABSTRACT

This invention relates to a device for liquid removal in particle-based preparative and analytical assays. The device utilizes a porous membrane to contain a liquid in the reaction chamber of the assay. The membrane enables the liquid to flow through once it comes in contact with a detachable absorbent pad. The combined use of the porous membrane and the absorbent pad allows for effective removal of the waste liquid by capillary force, thereby minimizing the carryover contamination caused by the residual liquid. The invention also relates to arrays comprising the device and assay methods using the device. It was possible to isolate DNA with high purity on a sieve-through platform using particle-based solid-phase extraction with the device. Particle-based ELISA was run on the sieve-through device to analyze proteins and cells with reduced background and greater signal-to-background ratio. In addition, a high-throughput potential of the sieve-through device with a 3×4 sieve-array that allowed for parallel processing of multiple samples has been found.

### TECHNICAL FIELD

The present invention generally relates to a sieve-through vertical flow device for efficient liquid exchange in particle-based assays, especially bioassays. A sieve-through platform that utilizes a porous membrane to sieve out the particles, and an absorbent pad to remove the waste liquid by capillary force is part of the inventive sieve-through device. The porous membrane is able to contain the liquid in the reaction chamber and also allows the waste liquid to flow through when it is brought into contact with the absorbent pad.

### BACKGROUND ART

Particle-based systems have been widely adopted in many preparative bioassays and analytical bioassays. There are several advantages associated with the particle-based systems. First, particles provide the solid substrate for molecule binding. Target molecules could non-specifically adsorb to the particles, such as DNA adsorption to silica particles.

Alternatively, they could specifically bind to the particles via the ligand-receptor interactions for complementary hybridization. For examples, antibody-conjugated particles could bind to target molecules in applications such as immunoassays. Oligonucleotide-conjugated particles are used in the DNA library preparation for high-throughput sequencing. Secondly, particles with unique properties, such as colour, size, and charge, are useful tags to label target molecules or cells. For example, fluorescent particles are commonly used as barcodes for multiplexed detection. Thirdly, particles have large surface-to-volume ratio, and therefore a small amount of particles would provide sufficient surface area for solid phase reactions. Last, particles are easily dispersed in the liquid, and easily separated from the solution phase by either centrifugal or magnetic force.

A typical particle-based assay requires several liquid exchange steps. After binding to target molecules, particles are first separated from the liquid phase by either centrifugal or magnetic force. Next, a liquid handling device, usually a pipette or a vacuum-loaded aspirator, is used to remove the waste liquid from the particles. After that, a new reagent is added, and the particles are re-dispersed. There are a number of issues associated with this liquid-exchange process that could potentially compromise the performance of particle-based assays. Firstly, it is difficult to completely remove the liquid from the particles. Because of surface tension, a small amount of liquid would stick to the particle surface. Moreover, the liquid would also get trapped at the interstitial space between particles. The residual liquid would significantly contribute to the contamination even after several rounds of washing. Such contamination is observed in the DNA isolated using silica particles, evidence by its abnormal 260/280 and 260/230 ratios. Secondly, while it is relatively easy to add the liquid to particles, retrieving the liquid from particles requires a higher level of hands-on skills. One needs to carefully position the liquid handling device, so that maximal amount of waste liquid could be removed without disturbing the particles. If too much liquid is left in the residue, it would lead to a high level of carry-over contamination. If particles are removed with the waste liquid by accident, it would result in a low yield or a low detection sensitivity. Accordingly the performance of particle-based systems is often compromised by the carry-over contamination caused by the residual reagents during the liquid-exchange process.

There is therefore a need to efficiently separate the particles of particle-based assays from the liquids used in various assay steps. A device that is able to effectively remove the waste liquid, thereby achieving a more efficient liquid exchange, as compared to the conventional process, and minimizing the carry-over contamination is therefore desired in the art.

### SUMMARY OF INVENTION

According a first aspect of the invention a device for liquid removal in particle-based assay systems comprising (a) a chamber with at least one opening for adding particles, liquids and optionally other materials and with at least one other opening for removing liquids from the chamber (b) a porous or sieve-like membrane, attached to the chamber covering the at least one opening for removing the liquid, to prevent the flowing of a liquid out of the chamber when not in contact with an absorbent pad, wherein the membrane has pores or holes with a diameter that is smaller than the particle diameter of the particles used in the assay; and (c) a membrane-detachable liquid absorbent pad that can be attached to the outer side of the membrane not facing the inner chamber has been made.

Advantageously, the device according to the invention can be used to effectively remove the waste liquid from particles. The capillary-driven liquid-exchange process by the absorbent pad is able to efficiently separate (“wipe off”) the waste liquid from the particles, thereby reducing any carry-over contamination. The absorbent pad may provide a passive pumping mechanism for simple fluidic handling. In contrast, the conventional liquid exchange by centrifugal or magnetic force would leave a large amount of liquid behind on the particle surface as a result of surface tension, leading to a high level of contamination.

The removal with the absorbent pad comprised in the device allows reactions to incubate for a predefined duration in the fluidic handling chamber, while the absorbent pad is detached from the membrane. It removes the waste liquid when the absorbent pad is brought into contact with the membrane. It further allows for passive fluidic pumping, which eliminates the use of bulky and expensive external fluidic control systems.

The embodiments of the invention advantageously provide a capillary-driven vertical flow platform for liquid exchange in particle-based systems. The platform, referred to by the name “sieve-through”, consists of a reaction unit and an absorbent pad (FIG. 1). The key feature of the sieve-through platform is the porous membrane that forms the bottom of the reaction chamber. Despite the fact that it is porous, the membrane is able to contain the liquid in the reaction unit due to surface tension. However, when the membrane is brought into contact with the absorbent pad, the capillary force provided by the absorbent pad would pull the liquid out of the reaction unit through the pores. The pores of the membrane are smaller than the diameter of the particles used in the assay. As a result, particles would be sieved out and retained on the membrane during the removal of the waste liquid through the pores, hence the name “sieve-through”.

According to a second aspect of the invention there is provided an array of devices according to the first aspect of the invention wherein the membranes of all or several devices can be contacted by a single absorbent pad or multiple sets of absorbent pads either simultaneously or at staggered sequence.

Advantageously, the array provides an embodiment of the invention wherein this embodiment has the potential for use in high-throughput analysis by presenting a sieve-array, which allows concurrent analysis of multiple samples in parallel. The sieve-through array may significantly improve the performance of particle-based systems.

According to a third aspect of the invention, there is also provided a preparative or quantitative assay method, comprising the steps of (a) providing a chamber with at least one opening for adding particles, liquids and optionally other materials and with at least one other opening for removing liquids from the chamber which is covered by a porous or sieve-like membrane; (b) filling particles and at least one liquid into the chamber together with a preparation of a sample material and optional reagents and/or other materials into the chamber; (c) performing a reaction in the chamber wherein the particles react or interact with a preparation of a sample material without any substantial flow of liquid through the membrane; (d) removing the reaction liquids and optionally dissolved by-products via the porous or sieve-like membrane by causing a flow through the membrane after contacting the membrane with an absorbent pad at the outer side of the membrane not facing the inner chamber; (e) optionally adding a liquid to the chamber; and (f) detecting or collecting the sample material that has reacted with the particle.

The method makes uses of the fluidic handling device according to the first aspect of the invention. Advantageously, the inventive method can be used to handle the liquid exchange in particle-based enzyme-linked immunosorbent assay (ELISA). The inventive method for liquid removal by “sieving” effectively allows for a removal of the waste liquid during ELISA, resulting in a low background. The method can be used in a broad variety of assays with liquid removal steps and is not limited to quantitative assays, but can also be used for preparatory assays. One embodiment therefore relates to a method wherein the particles interact with the sample material to form sample materials which are tagged by the particle by adherence or chemical binding. Advantageously, these tagged materials can be easily separated from the reaction solution and washed before further use.

According to a fourth aspect of the invention, there is provided a preparative or quantitative assay method, comprising the steps of (a) providing a chamber with at least one opening for adding particles, liquids and optionally other materials and with least one other opening for removing liquids from the chamber which is covered by a porous or sieve-like membrane; (b) filling sample material in particle form or immobilized on particles in a liquid preparation and optional reagents or fillers into the chamber; (c) removing the liquids and optionally dissolved substances via the porous or sieve-like membrane by causing a flow through the membrane after contacting the membrane with an absorbent pad at the outer side of the membrane not facing the inner chamber; (d) optionally adding a liquid to the chamber; and (e) detecting or collecting the sample material in particle form or immobilized on the particles. According to this method according to the invention sample materials that are already in particle form or tagged to particles can be separated and/or washed in a simple way. As such the method even allows analyzing “natural particles” such as cells. Advantageously, liquid removal or exchange is very simple.

### Definitions

The following words and terms used herein shall have the meaning indicated:

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “assay” refers to an investigative (analytic) procedure in laboratory medicine, pharmacology, environmental biology and molecular biology for qualitatively assessing or quantitatively measuring the presence, amount, or functional activity of a target entity (the analyte). Assay is used in a broad sense in this description and includes preparatory assay methods which are directed to removing the analyte from a preparation to determine its amount or to use it further.

As used herein, the term “bioassay” refers to a procedure for determining the concentration, purity, and/or biological activity of a substance (e.g., vitamin, hormone, plant growth factor, antibiotic, enzyme) by measuring its effect on an organism, tissue, cell, enzyme or receptor preparation compared to a standard preparation.

As used herein, the term “ELISA” refers to analytic biochemistry assay that uses a solid-phase enzyme immunoassay (EIA) to detect the presence of a substance in a liquid sample or wet sample.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value.

Unless specified otherwise, the terms “comprising” and “comprise”, and grammatical variants thereof, are intended to represent “open” or “inclusive” language such that they include recited elements but also permit inclusion of additional, not recited elements.

The word “substantially” does not exclude “completely” e.g. a composition which is “substantially not leaking” may be completely tight without any leakage. Where necessary, the word “substantially” may be omitted from the definition of the invention.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and subgeneric groupings falling within the generic disclosure also form part of the disclosure. This includes the generic description of the embodiments with a proviso or negative limitation removing any subject matter from the genus, regardless of whether or not the excised material is specifically recited herein.

### DETAILED DISCLOSURE OF EMBODIMENTS

Non-limiting embodiments of the invention will be further described in greater detail by reference to specific examples, which should not be construed as in any way limiting the scope of the invention.

According to a first aspect, there is provided a device for liquid removal in particle-based assay systems comprising (a) a chamber with at least one opening for adding particles, liquids and optionally other materials and with at least one other opening for removing liquids from the chamber; (b) a porous or sieve-like membrane, attached to the chamber covering the at least one opening for removing the liquid, to prevent the flowing of a liquid out of the chamber when not in contact with an absorbent pad, wherein the membrane has pores or holes with a diameter that is smaller than the particle diameter of the particles used in the assay; and (c) a liquid absorbent pad that can be attached to and detached from the outer side of the membrane not facing the inner chamber.

FIG. 1c shows one possible embodiment of the device. The chamber (1) has an opening for adding particles, liquids and optionally reagents from the top. On the bottom it has another opening for removing liquids from the chamber which is covered by a porous membrane (3). This membrane prevents the flowing of a liquid out of the chamber when not in contact with an absorbent pad. The membrane has holes of micro meter diameter which are smaller than the particles used in the chamber. The whole fluidic handling unit (2) can have support post for easier handling (4) for preventing contact of the membrane with other surfaces. The fluidic handling unit is combined with a detachable absorbent pad. If the liquid absorbent pad is attached to the outer side of the membrane the fluid can be removed from the chamber (1).

The chamber may be cylindrical as shown as an example in FIG. 1c, but is in no way limited to such geometric shape. The chamber volume and size are not critical features and can be varied widely. A chamber volume of about 200 to 400 μL may be mentioned which is suitable for a variety of assays. A special chamber volume that can be mentioned is about 300 μL, but 100 μL and 500 μL can also be mentioned. Chamber volumes from about 50 μL to 1 ml or 10 ml may be designed. The chamber may have a diameter of 0.1 to 2 cm, preferably 0.5 cm to 1.5 cm, but larger diameters of up to 5 cm may be use as well.

The flow of the liquid through the membrane may be passive (i.e., gravitational or capillary flow) or actively supported (flow resulting partly from the action of a flow pump, manual pressure, or vacuum). Preferably the flow is passive and driven by capillary forces upon contact of the absorbent pad with the membrane. The driving force can be the absorbent pad. The absorbent pad can have various geometrical shapes. A sheet shape may be particularly mentioned. The absorbent pad can be made from any material that has a sufficient water absorption capability. The material can be for instance comprise cellulosic fibers, such as the natural cellulosic fibers including cotton, ramie, jute, hemp, flax, and bagasse, and the synthetic cellulosic fibers, such as rayon and cellulose acetate, but is not limited thereto. In a preferred embodiment of this invention the absorbent pad material is made from hydrophilic materials, such as for instance from a list including Ahlstrom materials catalogue numbers 270 and 320, Schleicher & Schuell catalogue numbers 300 and 900 among others.

According to one embodiment the liquid can be removed from the chamber by vertical flow and the at least one opening for adding reagents is on top or at an upper section of the chamber and the at least one other opening for removing liquids from the chamber is at the bottom or at a lower section of the chamber. Preferably the at least one opening for adding reagents is on top of the chamber and the at least one other opening for removing liquids from the chamber is at the bottom of the chamber. The liquid can then be removed by a combination of gravitational and capillary forces. The liquid removal may be followed by adding another liquid for further reaction, storage or washing. In this regard the inventive device may be used for a liquid exchange in the assays.

According to one embodiment the membrane becomes permeable for the liquids upon contact with the liquid absorbent pad. Before contact with the pad the membrane may be able to contain the liquid in the fluidic chamber due to surface tension despite being porous or having holes. The difference in permeability and the resulting flow may be achieved by capillary forces from the pad. The membrane can be a non-sorbent porous (NSP) membrane. As a non-sorbent material the membrane may not substantially absorb or adsorb substantial amounts of the liquid from the chamber when the absorbent pad is not in contact with the membrane. Once in contact with the absorbent pad, the NSP membrane allows the liquid to substantially, preferably completely, flow through it, while retaining particles on the membrane surface for solid phase reaction or collection of the particles. The inventive sieve-through mechanism enables the complete removal of liquid from the solid phase (particles), which significantly reduces carry-over contamination.

According to one embodiment the chamber is a reaction chamber for performing a chemical or biochemical reaction in a liquid. It can therefore be used as part of a respective (bio)assay. Waste that can contaminate the particles after performing the reaction can be removed together with the solvent by using the inventive device. Preferably the reaction takes place while the absorbent pad is not attached to the membrane and the liquid held in the chamber. After a suitable definable reaction and hold-up time the absorbent pad can be brought in contact with the membrane. According to one embodiment of the invention the membrane prevents the liquid from flowing from the chamber with a hold-up time (defined as the duration that the liquid is contained in the chamber without substantially leaking through the membrane) of more than 5 minutes, preferably more than 10, 15, 30, 45 or 60 minutes. The hold-up time is defined by the liquids used. In one embodiment a liquid with high surface tension is used characterized by a maximum concentration of 5% (v/v) of a surface active ingredient (such as Tween), preferably nor more than 3.5%, 2%, 1%, 0.5% or 0.1%.

When the membrane is brought into contact with the absorbent pad, the capillary force provided by the absorbent pad pulls the liquid out of the chamber through the pores or holes. The pores or holes of the membrane are smaller than the diameter of the particles used in the assay. As a result, particles would be sieved out and retained on the membrane during the removal of the waste liquid through the pores, hence the name “sieve-through”. The membrane is preferably a microporous membrane. Typical pore or hole sizes are 0.05 to 50 μm, preferably 0.5 to 25 μm, more preferably 1 to 15 μm and most preferably 3 to 8 μm. Other pore and micro hole sizes that can be mentioned are 2, 4, 5, 6, 7, 9, 10 12 and 17 μm.

The membrane may be made from a non-absorbent polymer which makes the membrane non-sorbent and does not allow for leakage of the liquid through the membrane when the pad is detached. Polymers which may be used as the membrane material include, but are not limited to, polycarbonates, polyamides, modacrylic copolymers, styrene-acrylic acid copolymers, polysulfones, polyvinylidene fluoride, polyvinylfluoride, polychloroethers, thermoplastic polyethers, acetal polymers, polyacrylonitrile, polymethyl methacrylate, poly n-butyl methacrylate, polyurethanes, polyimides, polybenzimidazoles, polyvinyl acetate, aromatic and aliphatic polyethers, cellulose esters, epoxy resins, polyethylene, polypropylene, porous rubber, poly(ethylene oxides, polyvinylpyrrolidones, poly(vinyl alcohols), poly(sodium styrenesulfonate), polyvinylbenzyltrimethyl-ammonium chloride, poly(hydroxyethyl methacrylate), poly(isobutyl vinyl ether), polyisoprenes, polyalkenes, ethylene vinyl acetate copolymers, polyamides, and polyurethanes. Microporous or micro-hole containing polycarbonate materials may be preferred.

The liquid is preferably an aqueous medium, which may in addition to water comprise other liquids such as alcohols optionally together with buffers used in the assays, such as PBA (phosphate buffered saline containing bovine serum albumin) or PBS (phosphate buffered saline) in various mixtures. The liquid may be adjusted by mixing with other solvents and buffer to guarantee a significant hold-up time as needed for a reaction in the chamber. The device may be also adjusted to be used at different temperatures as needed for a reaction in the chamber; however, mostly room temperature (20 to 27° C.) may be chosen.

According to various possible embodiments the particle-based assay may be preparative or quantitative bioassay. It can be used for all assays where a solid phase (particle) needs to be separated from a liquid, such as separation of a reaction solvent with contaminants. The particle can be separated for collection thereof or for further washing and/or reaction steps to be performed in the chamber. In this regard the removal of liquid from the chamber can be performed once or several times as needed to run the assay with desired procedural requirements.

Depending on the assay and the procedure the particle is optionally functionalized and is selected from silica particles, silica-coated magnetic particles, polymeric particles, magnetic or superparamagnetic particles. Typical particles include commercially available magnetic beads, such as marketed by Thermo Fisher Scientific (Massachusetts, USA) in a line of magnetic beads under the brand name Dynabeads® or silica-coated magnetic beads, such as for instance Fe₃O₄ magnetic beads coated with a silicon dioxide (SiO₂) layer. Polymeric particles may also be used, for instance as solid phase for ELISA applications. The polymeric particles may be selected from polycarbonate, polypropylene, polyvinyl, nylon, nitrocellulose, polystyrene and maleic anhydride activated polystyrene particles. Polystyrene particles may be particularly mentioned. The particles are bigger than membrane pores or holes and may have an average diameter of are 0.1 to 100 μm, preferably 0.75 to 30 μm, more preferably 1 to 15 μm and most preferably 3 to 8 μm. Other sizes that can be mentioned are 2, 4, 5, 6, 7, 9, 10 12 and 20 μm.

According to a specific embodiment particles are deposited on the membrane or on the chamber inner walls. Such particles may be further functionalized for use in a bio reaction of the assay, such as for instance polystyrene particles with conjugated antibody (e.g. a capture antibody in a particle-based ELISA). Devices with deposited particles may be preferably used in ELISA applications. The reaction can be easily started by filling the sample together with liquids and other optional reagents or additives (e.g. buffer) into the chamber. The device is in this way prepared for a specific assay type and easy to handle. The liquid is removed or exchanged after the reaction via the porous or sieve-like membrane.

The other materials added in the first opening of the chamber may comprise reagent, sample preparations or other materials needed for the assay.

The use of the device according to the invention can be scaled up for higher throughput and parallel processing of multiple samples. From a singular device in its basic form, a scale up of the (reaction) chambers into an array of a large number of devices is possible. Potential array configuration such as 6, 12, 24, 48, 96, 384, or any other number of wells can be achieved as desired.

In this and similar array configurations, an array of devices wherein the membranes of all or several devices can be contacted by a single absorbent pad or multiple sets of absorbent pads either simultaneously or at staggered sequence according to the assay need is a second aspect of the invention. According to one embodiment of the invention an array of devices can be set up wherein the membranes of all or several devices can be contacted with one absorbent pad simultaneously to achieve a liquid removal from all device chambers substantially one single procedural step.

A 96-well plate format may be preferred for a high-throughput array. A chamber volume of about 200 to 400 μL is suitable for a variety of assays. A special chamber volume that can be mentioned is 300 μL, but 100 μL and 500 μL can also be mentioned. Existing lab automation support (e.g. transport system, liquid handling and machine vision) is available for the 96-well format. Fully automated high-throughput sieve-through vertical flow array can be made using the inventive devices. This may include automated sample and reagents dispensing, automated plate transport, automated absorbent pad deployment and exchange/disposal, and an automated elution process. The elution process in an array may be achieved via the use of positive pressure, vacuum, capillary pressure, centrifugal force, etc., or a combination of the above.

The device according to the invention can be made by known manufacturing techniques as exemplified in the examples. The chamber is preferably made of 3-D printed inert material. The material may be chosen from ABS plastic, PLA, polyamide (nylon), glass filled polyamide, stereolithography materials (epoxy resins), silver, titanium, steel, wax, photopolymers and polycarbonate, but its choice is not critical. The membrane can be glued, laminated or fused by heat to the chamber covering the opening. An array according to the invention wherein the array consists of a well plate wherein the membrane is attached to the bottom of the well plate by adhesive, double-sided tape, PDMS or thermal bonding of the membrane to the plate bottom is another embodiment of the invention. The membrane can also be attached during injection molding (for mass production). The membrane of the arrays is preferably a polycarbonate membrane with micrometer-size holes.

According to a third aspect of the invention a preparative or quantitative assay method has been found, comprising the steps of (a) providing a chamber with at least one opening for adding particles, liquids and optionally other materials and with at least one other opening for removing liquids from the chamber which is covered by a porous or sieve-like membrane; (b) filling particles and at least one liquid into the chamber together with a preparation of a sample material and optional reagents and/or other materials into the chamber; (c) performing a reaction in the chamber wherein the particles react or interact with a preparation of a sample material without any substantial flow of liquid through the membrane; (d) removing the reaction liquids and optionally dissolved by-products via the porous or sieve-like membrane by causing a flow through the membrane after contacting the membrane with an absorbent pad at the outer side of the membrane not facing the inner chamber; (e) optionally adding a liquid to the chamber; and (f) detecting or collecting the sample material that has reacted with the particle. The preferred sequence of steps is a), b), c), d), optionally e) and f).

The chamber of step a) may be the chamber that is part of the device according to the first aspect of the invention. The device as described in detail according to the first aspect of the invention may therefore be used in this method. The particles, liquids membranes are those as described above for the assays using the inventive device according to the first aspect of the invention.

The assay method can be a known particle-based assay method such as a particle based ELISA method. All particle-based ELISA formats may be used. An ELISA assay format which is a “sandwich” assay may be particularly mentioned. In this type of capture assay or “sandwich” assay the analyte to be measured is bound between two primary antibodies—the capture antibody and the detection antibody. The sandwich format is used because it is sensitive and robust and can be used in particle based ELISAs. The reagents are common reagents used in preparative or quantitative (bio)assays. In the case of a “sandwich” ELISA they may be a detector antibody.

In step b) the particles of the particle based assay are filled in the chamber of a device together with a liquid which may be the main reaction medium of the assay. A sample material is also filled in the chamber. The sample may be used in a preparation of any form that is commonly employed in the particle based assay and may include solvents, liquids, buffers, naturally occurring substances, additives and fillers. The preparation of the sample may be obtained from a living organism directly or by prior reaction or modification. The sample material may be a biological material, such as a cell, a chemical substance present or produced in a living organism, a biomolecule, a molecule present in a living organism, a biogenic substance or a chemical substance produced by a living organism. The biological material may have been reacted with a reagent (e.g. a detector antibody in an ELISA) before filling into the chamber. Optionally the reagent of the assay and other fillers or additives may be added together or in sequence according to the assay needs in step b).

In step c) a reaction is performed in the chamber wherein the particles react or interact with a preparation of a sample material without any substantial flow of liquid through the membrane. The particles remain in the chamber together with the liquid. The reaction is controlled by time and preferably run to achieve a full conversion of the sample material to the reaction product as needed for the assay. The reaction may be an antibody reaction, a covalent or other binding reaction, an enzymatic reaction, but is not limited to such reaction type. In another embodiment the sample material may be chemically bound or adhering to the particles. In a particle-based ELISA the reaction can for instance be the binding of the sample with a capture antibody on the particle.

In one embodiment the particles interact with the sample material to form sample materials which are tagged by the particle by adherence or chemical binding. This embodiment is especially well suited to tag cells and prevent their leakage through the membrane pores. Typical particles that can be mentioned include polystyrene, silica, sepharose or metal particles, including magnetic particles. The particles are bigger than the pores or holes in the porous or sieve-like material.

In step d) the reaction liquids and optionally dissolved by-products are removed via the porous or sieve-like membrane by causing a flow through the membrane after contacting the membrane with an absorbent pad at the outer side of the membrane not facing the liquid filled inner chamber. The flow can be caused by the same means as described above for the inventive device, e.g. by capillary forces. The absorbent pad is the same as described for the inventive device above. Preferably the liquid and all by-products of the reaction as well as contaminants interfering with the following assay steps are removed to a substantial degree, more preferably completely, together with the liquid. Optional step e) can be used to fill another liquid into the chamber for instance for the purpose of having a dispersion of the particles for detection or collection in step f). In another embodiment the chamber is filled with a washing liquid after step d) and thereafter this washing liquid is removed through the membrane by further contacting the membrane with the absorbent pad before performing to step e) or f). The washing liquid could be a typical washing liquid such as an optionally buffered aqueous solution, an organic solvent or mixtures thereof. These washing steps can be repeated 1 to 5 times, preferably 2 times.

In step f) the sample material that has reacted with the particle in step c) is detected or collected. In preparative assays the sample material is collected after binding or adhering to the particles. The sample material may be cleaved off or separated from the particles in the collection step for instance by using an acid or buffer solution. In quantitative assays the particles obtained after previous steps may be detected using the common detection means of a particle based assay. In an ELISA the detection may be based on the measuring of an enzyme activity. All detection methods are possible such as magnetic measurements, UV-VIS spectrometry, fluorescence measurements, etc. The detectable material may also be obtained by another step g) in which the collected sample material is reacted with a labeling substance. Examples for such labeling substances are antibodies conjugated to a fluorescent dye, fluorescent dyes, magnetic or optical markers etc.

In one embodiment of the method according to the third aspect of the invention the particles are polymeric particles functionalized with a labelled capture antibody and the preparation of the sample material comprises an enzyme-labeled detector antibody. Such method may be a particle-based “sandwich” ELISA method.

In another embodiment of the method the sample preparation comprises DNA and the particle is a magnetic particle. The method may be suited to collect DNA from a sample preparation in a preparatory assay.

In yet another embodiment of the method the sample preparation comprises mRNA and the particle is a poly(T) conjugated magnetic particle. The method may be suited to collect mRNA from a sample preparation in a preparatory assay.

In another embodiment of the inventive method the particles are first filled into the chamber in step b) together with a liquid, the liquid is removed and the particle deposited on the membrane and or/chamber walls before the sample material together with at least one liquid is filled in the chamber. The deposition of particles to the membrane and or/chamber walls can be achieved by typical drying steps after removal of the liquid. The drying can be achieved by air-drying or heating. According to yet another embodiment the particles can also be pre-deposited into the chamber by other means, such as for instance as a powder comprising the optionally pre-modified particle. The pre-deposited particles can remain in the chamber and stored for more than 7 days or even 12 months. The opening of the chamber can be sealed after depositing the particles to increase storage time.

The steps a) to f) can be performed at various temperatures. An ambient temperature of about 20 to 27° C. may be particularly mentioned. The concentration of particles, reagents and sample materials in the liquid depend on the assay and can be easily determined by the skilled person in the art from the studying of respective assay methods that use different liquid removal steps.

According to a fourth aspect of the invention a preparative or quantitative assay method has been found, comprising the steps of (a) providing a chamber with at least one opening for adding particles, liquids and optionally other materials and with at least one other opening for removing liquids from the chamber which is covered by a porous or sieve-like membrane; (b) filling sample material in particle form or immobilized on particles in a liquid preparation and optional reagents or fillers into the chamber; (c) removing the liquids and optionally dissolved substances via the porous or sieve-like membrane by causing a flow through the membrane after contacting the membrane with an absorbent pad at the outer side of the membrane not facing the inner chamber; (d) optionally adding a liquid to the chamber; and (e) detecting or collecting the sample material in particle form or immobilized on the particles. The preferred sequence of steps is a), b), c), d), optionally e) and f).

The chamber of step a) of this fourth aspect of the invention may be the chamber that is part of the device according to the first aspect of the invention. The device as described in detail according to the first aspect of the invention may therefore be used in this method. The particles, liquids membranes are those as described above for the assays using the inventive device. The assay method can be a known particle-based assay preparative method such as DNA or mRNA extraction method.

In step b) the sample material in particle form or immobilized on particles in a liquid preparation and optional reagents or fillers are filled into the chamber. The sample may be used in a preparation of any form that is commonly employed in the particle based assays and may include solvents, liquids, buffers, naturally occurring substances, additives and fillers. The preparation of the sample may be obtained from a living organism directly or by prior reaction or modification. The binding to a particle by conjugation or other chemical or physical bonding may be a chosen type of modification. The sample material may be a biological material, such as a cell, a chemical substance present or produced in a living organism (e.g. a protein), a biomolecule, a molecule present in a living organism, a biogenic substance or a chemical substance produced by a living organism. The biological material may have been reacted with a reagent (e.g. for instance with a magnetic particle) before filling into the chamber. Optionally the reagent of the assay and other fillers or additives may be added together or in sequence according to the assay needs in step b).

According to one embodiment the sample material may be a cell tagged with a particle.

In step c) the liquids and optionally dissolved by-products are removed via the porous or sieve-like membrane by causing a flow through the membrane after contacting the membrane with an absorbent pad at the outer side of the membrane not facing the liquid filled inner chamber. The flow can be caused by the same means as described above for the inventive device, e.g. by capillary forces. The absorbent pad is the same as described for the inventive device above. Preferably the liquid as well as contaminants derived from preparing the sample preparation and which are interfering with the following assay steps are removed to a substantial degree, more preferably completely together with the liquid. Optional step d) can be used to fill another liquid into the chamber for instance for the purpose of having a dispersion of the particles for detection or collection in step e). In another embodiment the chamber is filled with a washing liquid after step c) and thereafter this washing liquid is removed through the membrane by further contacting the membrane the absorbent pad before performing to step d) or e). The other liquid could be a typical washing liquid such as an optionally buffered aqueous solution, an organic solvent or mixtures thereof. These washing steps can be repeated 1 to 5 times, preferably 2 times.

In step e) the sample material in particle form or immobilized on the particles is detected or collected. In preparative assays the sample material is collected from the particles. The sample material may be cleaved off or separated from the particles in the collection step for instance by using an acid or buffer solution. In quantitative assays the particles obtained after previous steps may be detected using the common detection means of a particle based assay. All detection methods are possible, such as magnetic measurements, UV-VIS spectrometry, fluorescence measurements, etc. The detectable material may also be obtained by another step g) in which the collected sample material is reacted with a labeling substance. Examples for such labeling substances are antibodies conjugated to a fluorescent dye, fluorescent dyes, magnetic or optical markers etc.

In one embodiment of the method according to the fourth aspect the sample material absorbed or immobilized on particles comprises DNA or RNA and the particles are selected from silica beads or silica-coated magnetic beads. The method may be suited to extract/collect DNA from a sample preparation in a preparatory assay.

In another embodiment of the method the sample preparation comprises mRNA and the particles are selected from oligo(dT) magnetic beads.

The steps a) to f) of the method according to the fourth aspect of the invention can be performed at various temperatures. An ambient temperature of about 20 to 27° C. may be particularly mentioned. The concentration of particles, reagents and sample materials in the liquid depend on the assay and can be easily determined by the skilled person in the art from the studying of respective assay methods that use different liquid removal steps.

### EXAMPLES

The sieve-through platform and performed both preparative and analytical assays on the platform are characterized in the following examples which in no way limit the invention to the scope of the examples. A particle-based solid-phase DNA extraction is shown with reduced contamination of the isolated DNA. A particle-based enzyme-linked immunosorbent assay (ELISA) on the sieve-through platform is exemplified. The sieve-through platform effectively removed the waste liquid during ELISA, resulting in a low background. In addition, the ability of the inventive sieve-through platform to analyze “natural particles” such as cells using immunoassays is shown. Furthermore, a sieve-array that allows multiple reactions in parallel, demonstrating great potentials of the sieve-through platform for high-throughput applications is another example of the inventive device's use.

### Example 1: Device

### Device Prototype

The sieve-through platform consisted of a reaction unit and an absorbent pad (FIG. 1). The reaction unit was designed using the SOLIDWORKS (Dassault Systèmes, Villacoublay Cedex, France) and prototyped using the Stratasys 3D printer (Stratasys, Rehovot, Israel) (FIGS. 1a and 1b). The reaction unit was comprised of a reaction chamber and two support posts. A piece of polycarbonate membrane with micron-sized pores was glued to form the bottom of the reaction chamber (FIG. 1c). A wide selection of porous membranes of various pore sizes was commercially available (Nucleopores®, Sigma-Aldrich, Missouri, USA). The two support posts would hold the membrane in suspension so that the membrane was not in contact with any surface. The absorbent pad (Ahlstrom Filtration, Helsinki Finland) was cut into desired dimensions using a CO₂ laser cutter (Epilog Laser, Colorado, USA). The absorbent pad was kept apart from the reaction chamber, and was only brought into contact with the membrane during the liquid-exchange step. For ELISA on the sieve-through platform, particles with surface-conjugated antibodies were pre-stored on the porous membrane of the reaction chamber and sealed with the aluminum foil (FIG. 1b). FIGS. 1d and 1e illustrate polystyrene particles of 5-μm (FIG. 1d) and 10-μm (FIG. 1e) diameters on the membrane with 3-μm pore.

### Device Characterization

To measure the flow rate through the membrane, 200 μL of liquid was added to the reaction chamber. The reaction unit was then placed on the absorbent pad. The time taken for all the liquid to flow through the membrane was recorded. The flow rate was calculated and expressed in terms of volumetric flux with a unit of μL/cm²·s.

To measure the hold-up time, 200 μL of liquid was added to the reaction chamber. The reaction unit was placed on a horizontal surface apart from the absorbent pad. The time taken for the liquid to leak out from the reaction chamber through the membrane was recorded. The measurement was stopped if no leakage was observed after 1 hour.

The most crucial component of the sieve-through device is the porous membrane. The membrane must contain the liquid in the reaction chamber for a sufficiently long duration, and it should also allow the liquid to flow through it when it is brought into contact with the absorbent pad.

The pore size had significant influence on the flow rate. For all three buffers tested, including water, 5% (w/v) BSA in 1×PBS, and 70% (v/v) ethanol, the flow rate increased with increasing pore size (FIG. 2a). The properties of the liquid would affect the flow rate too. As the concentration of glycerol in the solution was increased, the solution became more and more viscous. As a result, the flow rate decreased with increasing glycerol concentration (FIG. 2b). In addition, the polarity of the solvent would also have an effect on the flow rate. Since the membrane was hydrophilic, polar solvent would flow through the membrane more easily. As the ethanol concentration in the water increased, the polarity of the solvent decreased, resulting in decreasing flow rate (FIG. 2c). Surprisingly, the surface tension of the liquid did not affect the flow rate. No significant change in the flow rate was observed with varying concentration of Tween 20 over 5 orders of magnitude (FIG. 2d). However, the surface tension of the liquid greatly changed the hold-up time, defined as the duration that the liquid was contained in the reaction chamber without leaking through the membrane. Going beyond the hold-up time, the liquid would leak through the membrane without the assistance of the absorbent pad. As shown in FIG. 2e, as the surface tension of the liquid decreased (i.e. increasing Tween 20 concentration), the hold-up time also decreased. For 10% (v/v) Tween 20 on the membrane with 8-μm pores, the liquid would leak through the membrane within ˜25 sec. For 0.1% Tween 20 on the membrane with 3-μm pores, there was no leakage observed within 1 h. Typical concentration of Tween 20 is less than 0.1% in commonly used buffers. Therefore, the membrane was able to hold the buffer in the reaction chamber for sufficient reaction time.

### Example 2: Isolating DNA with High Purity Using the Device

### DNA Extraction

Human genomic DNA (gDNA) was extracted using Qiagen Biosprint 15 blood kit (Qiagen, Venlo, Netherlands). All reagents were prepared according to manufacturer's instruction. 400 ng of human gDNA (Promega, Wisconsin, USA) in 20 μL water was first mixed with 20 μL of buffer AL, 20 μL of isopropanol alcohol and 2 μL of magnetic particles. The mixture was incubated in the reaction unit with the membrane with 3-μm pores for 10 minutes at room temperature. After the incubation, the liquid was removed by placing the reaction unit on the absorbent pad. The waste liquid would flow through the porous membrane and get absorbed by the absorbent pad. The washing process was done by adding the washing buffer to the reaction chamber and subsequently removing the waste washing buffer through the membrane using the absorbent pad. The particles were washed once with 50 μL of buffer AW1, and twice with 50 μL of buffer AW2. In the end, 20 μL of water was added to elute DNA from the particle surface.

For comparison, DNA was also isolated in the microcentrifuge tube using the same protocol. To exchange liquid in the microcentrifuge tube, the tube was placed on a magnetic stand (Thermo Fisher Scientific, Massachusetts, USA). The particles were pulled to the side wall of the tubes by the magnetic force, and a pipette was carefully inserted into the tube to remove as much waste liquid as possible. In the end, the DNA was also eluted in 20 μL of water.

To extract DNA from whole cells, 20 μL of sample containing various amount of cells were mixed with 20 μL of buffer AL, 20 μL of isopropanol, 2 μL of proteinase K and 2 μL of magnetic particles. After removing the waste liquid, the particles were washed once with 50 μL of buffer AW1, and twice with 50 μL of buffer AW2. The isolated DNA was eluted in 20 μL of 5 mM Tris buffer. The eluent was collected by pressurizing the reaction chamber with a syringe, which forced the solution through the membrane into a container.

To evaluate the purity of the isolated DNA, the 260/280 and 260/230 absorbance ratios were measured using the Nanodrop ND-1000 UV-Vis spectrometer (Thermo Fisher Scientific, Massachusetts, USA). The concentration of the isolated DNA was measured using the PicoGreen assay (Thermo Fisher Scientific, Massachusetts, USA). To do so, the eluted DNA was first diluted 50 fold in 1×Tris EDTA (TE) buffer (pH=8), and the stock PicoGreen reagent was diluted 200 folds in the same buffer. Next, the diluted DNA and the PicoGreen reagent were mixed at 1:1 volume ratio and incubated in the dark for 10 minutes. The DNA concentration was determined by measuring the fluorescent intensity using the Nanodrop ND-3300 fluorospectrometer (Thermo Fisher Scientific, Massachusetts, USA).

Isolating DNA with High Purity on the Sieve-Through Platform

It has been found that the DNA isolated using silica particles is heavily contaminated by the chemicals in the binding buffer, evidenced by the poor 260/280 and 260/230 ratios (Sambrook, J.; Russell, D. W. Molecular Cloning: A Laboratory Manual; Cold Spring Harbor Laboratory Press, 2001). The sieve-through platform device is able to obtain high-purity DNA by effectively removing the waste liquid during the binding and washing steps, thereby reducing the carry-over contamination.

Compared to the DNA isolated using the conventional particle-based procedure in the microcentrifuge tube, the purity of the DNA isolated on the sieve-through platform was significantly higher (Tab. 1). For DNA to be considered pure, the ideal 260/280 ratio was 1.8, and the ideal 260/230 ratio was 2.0-2.2. The input DNA, which also served as the control, had a 260/280 ratio of ˜1.96, and a 260/230 ratio of ˜1.77, which indicated reasonably good quality. The DNA isolated on the sieve-through platform had a 260/280 ratio of ˜1.80 and a 260/230 ratio of ˜1.67, which were close to those of the control. In comparison, the DNA isolated using the conventional approach in a microcentrifuge tube had a very high 260/280 ratio (˜2.46) and a very low 260/230 ratio (˜0.32). Both ratios deviated significantly from the optimal range, suggesting poor purity of the isolated DNA. As the contamination would influence the absorbance at 260 nm and lead to an over-estimation of the DNA concentration, the DNA concentration was best estimated using the PicoGreen assay. The DNA recovery yield on the sieve-through platform was ˜75%, and the DNA recovery yield obtained using the conventional approach is ˜69%. Both the sieve-through platform and the conventional approach used the same reagents to isolate DNA by particle-based solid-phase extraction. However, it was possible to obtain DNA with much higher purity on the sieve-through platform. The high purity directly resulted from the ability of the sieve-through platform to effectively remove the waste liquid and achieve efficient liquid exchange during the solid-phase extraction. It is believed that the sieve-through device could significantly improve the performance of many existing particle-based preparative assays.

[Table 1] shows the purity and recovery yield of DNA isolated on the sieve-through platform and in the microcentrifuge tube.

TABLE 1

| | | | Yield |
|---|---|---|---|
| Absorbance | 260/280 | 260/230 | (%) |
| Control | 1.96 ± 0.05 | 1.77 ± 0.09 | — |
| Sieve- | 1.80 ± 0.10 | 1.67 ± 0.07 | 75.5 ± 8.5 |
| Through | | | |
| Conventional | 2.46 ± 0.23 | 0.32 ± 0.12 | 69.0 ± 10.4 |

A silica particle-based DNA isolation from DLD-1 cell line using both the sieve-through platform and the conventional procedure in the microcentrifuge tube were also performed.

A 10-fold serial dilution of cells ranging from 20 to 20000 cells was used. Same as the previous case, the DNA isolated on the sieve-through platform had higher purity compared to the DNA isolated in the microcentrifuge tube (FIG. 3a and Table 2). The DNA isolated in the microcentrifuge tube was heavily contaminated and displayed an abnormal absorption spectrum, which resulted in a small 260/230 ratio. The contaminants included chaotropic agents and organic solvent, which would inhibit PCR amplification efficiency. The isolated DNA was then analyzed with real-time quantitative PCR. A set of primers and a Taqman probe that targeted the GAPDH gene was used to amplify the DNA (please refer to Supporting Information Table S2 for primer, probe and synthetic target sequences). Regardless the input cell number, the DNA samples isolated using the sieve-through platform exhibited lower threshold cycle (Ct) number (FIG. 3b). On average, the Ct value for the DNA isolated on the sieve-through platform was ˜2 cycles smaller, indicating higher DNA purity and/or higher DNA yield.

[Table 2] shows the 260/280 and 260/230 absorbance ratios of DNA isolated from HCT-116 cells (n=20,000) on the sieve-through platform and in the microcentrifuge tube.

TABLE 2

| | 260/280 | 260/230 | |
|---|---|---|---|
| Sieve- | 2.15 ± 0.02 | 2.08 ± 0.09 | Sieve-through |
| through | | | |
| Conventional | 2.52 ± 0.44 | 0.72 ± 0.32 | Conventional |

An mRNA isolation on the sieve-through platform using poly(T) conjugated particles was also performed.

### DLD-1 Cell Culture

DLD-1 cells (ATCC® CCL-221™) were purchased from ATCC (Virginia, USA). DLD-1 cells were seeded at a density of 5000 cells per cm2 into a T-75 flask and expanded in complete RPMI-1640 medium (ATCC 30-2001) containing 10% fetal bovine serum (FBS) (ATCC 30-2020) and 1% penicillin-streptomycin. Media changes were carried out every 2 days until the cells reached confluency. Once the DLD-1 cells reached about 90% confluency, they were harvested using 0.25% (w/v) Trypsin-0.53 mM EDTA solution. The cells were then pelleted by centrifuging at 125 g for 5 minutes. The supernatant was removed and the cell pellet was rinsed in 1×DPBS and centrifuged again. The supernatant was removed and the cell pellet was lysed in lysis buffer from the Dynabeads® mRNA DIRECT™ Kit (Thermo Fisher Scientific, Massachusetts, USA) for mRNA extraction.

### Example 3: Isolating mRNA

mRNA was isolated using poly(T) conjugated magnetic particles (Dynabeads® mRNA DIRECT™ Kit, Thermo Fisher Scientific, Massachusetts, USA). Cell pellets were incubated with 50 μL of lysis buffer and 10 μL of oligo (dT)25 functionalized magnetic particles. After incubation, the mRNA with poly(A) tails hybridized to the poly(T) conjugated magnetic particles. The waste liquid was removed through the membrane using an absorbent pad. Next, the particles were washed four times with 50 μL of washing buffer A, and once with 50 μL of washing buffer B by adding the buffer to the reaction chamber and removing the buffer through the membrane using an absorbent pad. The isolated mRNA was eluted in 10 μL of elution buffer at 70° C. The eluent was collected by pressurizing the chamber with a syringe, which forced the solution through the membrane into a container.

For comparison, we also performed mRNA isolation in the microcentrifuge tube according to the protocol suggested by the manufacturer. The liquid exchange was carried out by placing the microcentrifuge tube on a magnetic stand, which immobilized the particles to allow the removal of waste liquid. The particles were washed twice with 600 μL of washing buffer A, and 300 μL of washing buffer B in this case. All other conditions were the same.

It was possible to achieve high mRNA isolation efficiency as suggested by quantitative reverse transcriptase PCR (FIG. 4a). The performance of sieve-through platform was only slighter better than the conventional process performed in the microcentrifuge tube (FIG. 4b). Since the buffer used for mRNA isolation did not contain high concentrations of chaotropic agents or organic solvent, the carry-over contamination would not inhibit PCR.

### Example 4: ELISA Using Sieve-Through Device

### Method: Particle-Based ELISA

All reagents were purchased from Sigma-Aldrich unless otherwise stated. A model ELISA assay that detected the α-Fetoprotein (AFP) was used to demonstrate ELISA on the sieve-through platform device. The capture antibody (Arista Biologicals, Pennsylvania, USA) was labeled with biotin using the Lightning-Link® biotin kit (Innova Biosciences, Babraham, Cambridge, United Kingdom), and the detector antibody (Thermo Fisher Scientific, Massachusetts, USA) was labeled with horseradish peroxidase (HRP) using the Lightning-Link® HRP kit (Innova Biosciences, Babraham, Cambridge, United Kingdom). Streptavidin-conjugated polystyrene particles of 5-μm and 10-μm diameter (Bang's Laboratories, Indiana, USA) were washed once by pelleting the particles with 1200 g centrifugal force for 10 minutes and re-suspending the pellet in 100 mM phosphate buffer (pH=7.4). The desired amount of biotinylated capture antibody was diluted to 200 μL with 100 mM phosphate buffer (pH=7.4) and mixed with 200 μL of 1% (w/v) washed polystyrene particles. The mixture was then incubated on a rotator for 1 hour. After that, the particles were washed once and re-suspended in 400 μL of 100 mM phosphate buffer (pH=7.4) to a final concentration of 0.5% (w/v). For each reaction, 5 μL of antibody-conjugated polystyrene particles were mixed with 50 μL of 1× phosphate buffered saline (PBS) (First Base Technology, Singapore) supplemented with 5% (w/v) bovine serum albumin (BSA) (GE Healthcare, Connecticut, USA). The mixture was added to the reaction chamber with the membrane with 3-μm pores. For whole blood samples, the membrane with 5-μm pores was used. The particles conjugated to capture antibodies were allowed to dry on the membrane by removing the liquid using the absorbent pad. After that, the reaction chamber that contained the pre-dried particles was sealed with aluminum foil (FIG. 1b) and stored in low humidity until use.

To measure AFP, the HRP-labeled detector antibody was first diluted to 8 μg/mL in 1×PBS supplemented with 5% (w/v) BSA, and 5 μL of the diluted detector antibody was mixed with 50 μL of sample. The aluminum foil was peeled off, and the sample mixture was then added to the reaction chamber and incubated for 10 minutes. After that, the waste liquid was removed through the membrane using the absorbent pad. The polystyrene particles were washed twice with the washing buffer consisting of 1×PBS supplemented with 0.05% (v/v) Tween 20. Next, 50 μL of 1-Step™ ultra TMB-ELISA substrate solution (Thermo Fisher Scientific, Massachusetts, USA) was added to the reaction chamber and allowed to develop for 15 minutes. In the end, 50 μL of 0.16 M sulfuric acid was added to stop the reaction. The AFP concentration was determined by measuring the absorbance of the developed TMB using the Nanodrop ND-1000 UV-Vis spectrometer.

For comparison, the particle-based ELISA was also performed in the microcentrifuge tube. All reagents were the same as the ones used on the sieve-through platform. 5 μL of 0.5% (w/v) capture antibody-conjugated polystyrene particles were mixed with 40 μg of HRP-labeled detector antibody and 50 μL of sample in in 1×PBS supplemented with 5% (w/v) BSA. To exchange buffer, the particles were pelleted by centrifugation at 1200 g force for 10 minutes. The supernatant was then removed, and the washing buffer was added. In the end, the reaction was developed with 50 μL of TMB for 10 minutes, and stopped with 0.16 M sulfuric acid

### ELISA on the Sieve-Through Platform

The sieve-through platform is able to prevent the carryover contamination caused by residual liquid. This would allow a low background to be achieved for ELISA applications.

Compared to the particle-based ELISA performed in the microcentrifuge tube, the background was significantly lower on the sieve-through platform (FIG. 5a). Furthermore, the signal from positive samples was lower in the microcentrifuge tube, possibly due to the loss of particles during the liquid exchange. A 2-fold serial dilution of AFP on the sieve-through platform using the particle-based ELISA was successfully quantified (FIG. 5b). AFP was an important tumor marker commonly used for the diagnostics of liver cancer. The standard curve was fit appropriately with the 4-parameter logistic function with an R-square value of >0.99 (FIG. 5b inset). The particles used in this experiment have a diameter of 5 μm and the membrane has a pore size of 3 μm.

The effect of particle size on the ELISA was also examined. Particles of 5 μm and 10 μm in diameter were used to analyze the same serial dilution. Results from both particles agreed reasonably well with each other, suggesting that particle size did not have a strong influence on the ELISA (FIG. 5b). However, the amount of capture antibodies on the particles would affect the outcome of the ELISA. With 2 μg of capture antibody per mg of particles, the dynamic range of the assay would cover the entire serial dilution. If the loading of capture antibody was reduced to 0.8 μg per mg of particles, the signal decreased significantly for samples containing high concentration of AFP (FIG. 5c). In fact, the signal would decrease with increasing AFP concentration due to the “Hook's effect”.

The sieve-through platform was able to perform ELISA directly with whole blood samples. AFP was spiked into the whole blood, which was reconstituted by mixing packed red blood cells and cultured white blood cell line (Jurkat cells) with serum. The reconstituted blood contained 800 white blood cells per 1 μL of blood with a hematocrit of 45%. 10 μm-sized particles and membrane with 5-μm pores were used. Red blood cells were able to flow through this membrane. Although white blood cells might remain on the membrane, they are colorless and did not interfere with the signals. The results obtained from whole blood samples matched well with the results from samples in the buffer, suggesting that the sieve-through platform was capable of detecting targets directly from whole blood (FIG. 5d).

### Example 5: ELISA Using Sieve-Through Device

### Method: CD4+ Cell Count

CD4+ Jurkat cells (ATCC, Nevada, USA) were cultured in ATCC-formulated RPMI-1640 medium supplemented with 10% FBS (Thermo Fisher Scientific, Massachusetts, USA). The culture was maintained at 37° C. in 5% CO₂ environment. Fresh medium was added to keep the cell density below 1×106 viable cells/mL. Anti-CD4 antibody (Abcam, Cambridge, United Kingdom) was labeled with HRP using the Lightning-Link® HRP kit (Innova Biosciences, Babraham, Cambridge, United Kingdom). 40 ng of the labeled antibody was mixed with cells in 100 μL of 1×PBS supplemented with 1% (w/v) BSA, and incubated at room temperature for 10 min. The cells were then added to the reaction chamber. Next, the waste liquid was removed using the absorbent pad, leaving only the cells on the membrane. The pore size of the membrane was 3 μm for cells in the buffer, and 5 μm in the case of whole blood samples. Subsequently, the cells on the porous membrane were washed twice with 50 μL of 1×PBS supplemented with 0.05% (v/v) Tween 20. After that, 50 μL of 1-Step™ ultra TMB-ELISA substrate solution was added and developed for 15 min. At the end, the reaction was stopped using 0.16 M sulfuric acid. The cell count was determined by measuring the absorbance of the developed TMB substrate.

The cell count was also performed with the tagging particles. In such an event, 1 μL of 1% (w/v) anti-CD45 Dynabeads (Thermo Fisher Scientific, Massachusetts, USA) was added to the sample to capture the cells. The rest of the procedures remained the same.

### CD₄₊ Cell Count on the Sieve-Through Platform Device

The sieve-through platform operates by separating particles from the liquid based on their size using the porous membrane as the sieve. The same strategy is applicable to naturally existing particles such as cells.

As a proof of concept, a CD₄₊ cell count on the sieve-through platform device was done. CD₄₊ T-cell count was an important marker of the immune system, and was often used for the prognosis of individuals diagnosed with AIDS. To perform CD₄₊ cell count on the sieve-through platform device, cells were first tagged with HRP-labeled anti-CD₄₊ antibodies. The cells were then separated and washed on the sieve-through platform. Next, the cell quantity was determined by measuring the TMB signal. With increasing amount of input CD₄₊ cells, a corresponding increase in the absorbance was observed (FIG. 6a). The signal plateaued for samples with a high cell count.

The effect of membrane pore size was tested. Membranes with different pore size (1 μm, 3 μm, 5 μm and 8 μm) were used to quantify the CD₄₊ cells. For all four types of membranes, the signal decreased with decreasing cell input (FIG. 6b). The signals from the 3-μm and 5-μm membrane were at about the same level. The signals from the 8-μm membrane were consistently the lowest because the pore size was too large to retain the cells in the reaction chamber. The signals from the 1-μm membrane were also lower than those from the 3-μm and 5-μm membrane. This result was a bit counter-intuitive. One would expect the membrane with smaller pore size to capture more cells, hence resulting in higher signals. The small pore size may have induced higher shear stress that mechanically lysed the cells, leading to the low cell count.

By introducing tagging particles to the cells, the cell count on the sieve-through platform was significantly improved. As a result, it was possible to quantify the cells with a wider dynamic range and a better linearity (FIG. 6c). The tagging particles recognized CD₄₅ on the cell surface. They did not interfere with the target CD₄₊ antigen. Although they were larger than the pores, cells were highly deformable and might squeeze through the pores. The tagging particles, on the other hand, were rigid and unable to squeeze through the pores. As a result, the binding of the tagging particles to the cells would make it more difficult for the cells to squeeze through the membrane (FIG. 6d). It is noted that the pores on the membrane sometimes overlapped to form a large pore (FIG. 6e), and cells could get through those pores more easily. The tagging particles increased the size of the cells and prevented them from getting through those overlapping pores. Furthermore, the tagging particles captured the lysed cell membranes. These lysed cell membranes retained on the sieve-through platform would generate signal.

The tagging particles were applied to measure CD₄₊ cells in whole blood on the sieve-through platform device. However, the whole blood strongly interfered with the cell count. Although a positive correlation between the TMB absorbance and the cell count was observed, the signal was weaker than that for the CD₄₊ cells in 1×PBSB buffer (FIG. 6f). We speculated that the presence of a large amount of red blood cells would block the pores and induce a higher shear stress, lysing more CD₄₊ cells. In addition, the presence of red blood cells might interfere with the binding of antibodies to CD₄₊ cells.

### Example 5: High-Throughput Sieve-Through Vertical Flow Platform

### Prototype

The Sieve-Through Vertical Flow Platform Device can be scaled up for higher throughput and parallel processing of multiple samples. From a singular reaction well in its basic form, the reaction chambers can be scaled up into an array of a large number of wells. Potential array configuration such as 6, 12, 24, 48, 96, 384, or any other number of wells can be achieved as desired. In this array configuration, a sheet of absorbent pad or multiple sets of absorbent pads can be used for liquid waste removal from the multiple reaction chambers either simultaneously or at staggered sequence according to assay need.

For the prototype device described below, a 96-well plate format has been chosen for the high-throughput platform as its working volume of 300 μL is suitable for a variety of assays. Moreover, there are many existing lab automation support (e.g. transport system, liquid handling and machine vision) available for the 96-well format. A fully automated high-throughput sieve-through vertical flow platform processing system may be build. This will include automated sample and reagents dispensing, automated plate transport, automated absorbent pad deployment and exchange/disposal, and automated elution process. The elution process can be achieved via the use of positive pressure, vacuum, capillary pressure, centrifugal force, etc., or a combination of the above.

For the prototype device, a bottom-less 96-well plate is used, and a porous membrane (e.g. polycarbonate membrane with 3-μm pores) is attached to the bottom of the well plate to form the well bottom. Several potential methods are available for membrane attachment to the bottom of the well plate or device, for example by adhesive, double-sided tape, polydimethylsiloxane (PDMS) or thermal bonding of membrane to plate bottom.

A thermal bonding method has been chosen for the fabrication of prototype device. A polycarbonate membrane has a higher glass transition temperature Tg (147° C.), as compared to the polystyrene 96-well plate (90° C.). Thus, the membrane is not damaged during the thermal bonding process by placing it together with the 96-well plate on a hot-plate at a temperature range of 110 to 120° C. The thermal bonding time required may vary depending on how quickly sealing is desired. Generally, 10 to 20 minutes may be sufficient with the application of a light pressure from above the plate. FIG. 7 illustrates the fabrication process of high-throughput sieve-through vertical flow platform.

A number of applications are suitable for the high-throughput sieve-through the made vertical flow platform. In particular, one of the applications is nucleic acid extraction. The protocol for nucleic acid extraction using the prototype device is as follows:
1) First, 500 k cells (e.g. from HCT116 colorectal cancer cell line) are lysed using a cell lysis buffer.
2) The cell lysate is incubated with beads (silica beads or silica-coated magnetic beads for total DNA, RNA extraction; poly (dT) conjugated magnetic beads for mRNA extraction) for 10 minutes at room temperature. This step results in the adsorption of nucleic acids to beads.
3) The cell lysate containing nucleic acid adsorbed-beads is introduced into a reaction well of the high-throughput sieve-through vertical flow platform.
4) An absorbent pad is brought into contact with the membrane under the reaction well. The cell lysate waste liquid is absorbed and removed through the porous membrane by the capillary force exerted by absorbent pad fibers. Only the waste liquid is removed; the beads being larger than membrane pores would remain in the reaction chamber.
5) A wash buffer is introduced into the reaction well.
6) An absorbent pad is brought into contact with the membrane under the reaction well. Thus, waste liquid is absorbed and removed through the porous membrane.
7) Washing steps 5 to 6 can be repeated for an additional number of times (typically twice).
8) A small volume of elute solution e.g. 50 μL of 10 mM Tris-HCl, nuclease-free water or other suitable buffer is added into the reaction well, and allowed to incubate for a period of time (e.g. 10 minutes). The reduced ionic content introduced by elute solution dissociates nucleic acid from beads.
9) The elute solution containing nucleic acid is eluted from reaction well into a collector for downstream processing. The potential methods of elution include pressurized air purge, centrifugation, vacuum evacuation, absorption by a pad in paper-based assays, aspiration from the above membrane, etc.

Nucleic acid extraction with the high-throughput sieve-through vertical flow platform has been validated with mRNA extraction from 500 k HCT116 cells. Reagents used include poly (dT) conjugated magnetic beads, standard wash buffers, and 10 mM Tris-HCl elution buffer (e.g. the reagents found in typical commercial kits such as DynaBeads mRNA Extraction kit). Centrifugation at 4000 rpm for 10 min (Eppendorf 5810R) is used for elution. In the elution process, the high-throughput sieve-through vertical flow platform is placed above and secured to a conventional 96-well plate. Both the platform and the conventional 96-well plate are secured together with the respective wells aligned, thus facilitating collection of elute into the conventional 96-well plate during centrifugation.

The mRNA extraction performance of the high-throughput sieve-through vertical flow platform is compared with that of commercial DynaBeads (see FIGS. 8 to 11). The mRNA quality and quantity were measured using spectrophotometry (NanoDrop ND2000), it was found that the mRNA extracted using the high-throughput sieve-through vertical flow platform is superior or comparable to that of DynaBeads.

FIG. 8 shows a significantly higher absorbance at the 260 nm for mRNA extracted using the high-throughput sieve-through vertical flow platform, as compared to DynaBeads. A comparable 260/280 absorbance ratio was attained with both methods.

FIG. 9 shows a significantly higher concentration for mRNA was extracted using the high-throughput sieve-through vertical flow platform, as compared to DynaBeads.

FIG. 10 shows a lower elute volume recovered by the high-throughput sieve-through vertical flow platform (via centrifugation), as compared to DynaBeads (via aspiration by pipette). The elute volume recovery can be further optimized using the centrifugation approach, or an alternative approach such as using pressurized air purge, vacuum evacuation and aspiration.

FIG. 11 shows that a higher quantity of mRNA was extracted using the high-throughput sieve-through vertical flow platform, as compared to DynaBeads. The absolute quantity of mRNA extracted by the sieve-through can be further optimized as described above through increasing the elute volume.

### Sieve-Array

The sieve-through platform can be easily scaled up without adding complexity to the fluidic handling mechanism. As shown in FIG. 12a, sieve-through reaction units were aligned in an array that allowed to perform multiple reactions in parallel. The liquid-exchange process on the sieve-array took place at the same time using a large piece of absorbent pad that covered all the reaction units. It was more convenient to handle an array than individual reaction units when analyzing a large number of samples. Furthermore, the sieve-array also reduced the time required to perform the liquid-exchange through parallelization.

As shown in FIG. 12b, a serial dilution of AFP was measured on the sieve-array concurrently. The TMB signal decreased with decreasing AFP concentration. Although it was only demonstrated that a 3×4 array is possible, sieve-arrays of higher density would be plausible by reducing the size of the reaction units and the spacing in between. The sieve-through platform may be used in many high-throughput applications.

### DESCRIPTION OF DRAWINGS

The accompanying drawings illustrate a disclosed embodiment or reaction scheme and serve to explain the principles of the disclosed embodiments. It is to be understood, however, that the drawings are designed for purposes of illustration of examples only, and not as a limitation of the invention.

FIG. 1 shows an inventive sieve-through device. (a) Picture of the sieve-through prototype. (b) The sieve-through reaction unit sealed with aluminum foil. (c) Schematic illustration of different components of the sieve-through platform. (d) 5-μm particles on the membrane with 3-μm pores. (e) 10-μm particles on the membrane with 3-μm pores. (d) and (e) are false-colored scanning electron microscopy (SEM) images.

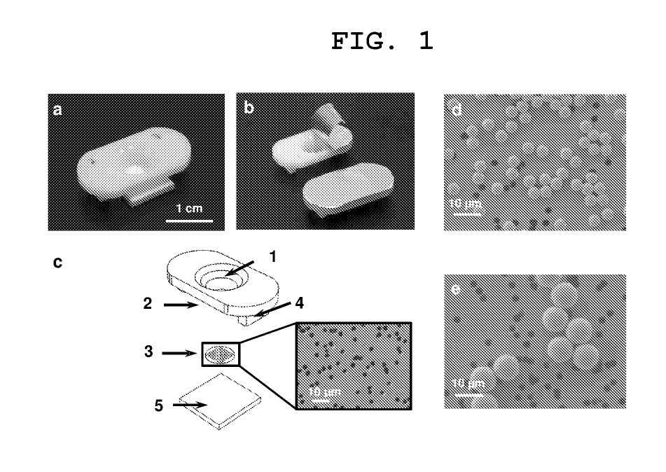

The schematic illustration of FIG. 1 c shows the features of the device: 1—fluidic handling chamber; 2—fluidic handling unit; 3—porous membrane; 4—optional support post; 5—absorbent pad.

FIG. 2 shows flow characteristics of the sieve-through device. The effect of (a) the membrane pore size [water is left column], (b) the viscosity of the liquid, (c) the polarity of the solvent, and (d) the surface tension of the liquid on the flow rate through the membrane, and (e) The effect of the liquid surface tension on the hold-up time in seconds. The white region indicates no leakage in 3600 sec; the greyscales according to the seconds are top down in the figure.

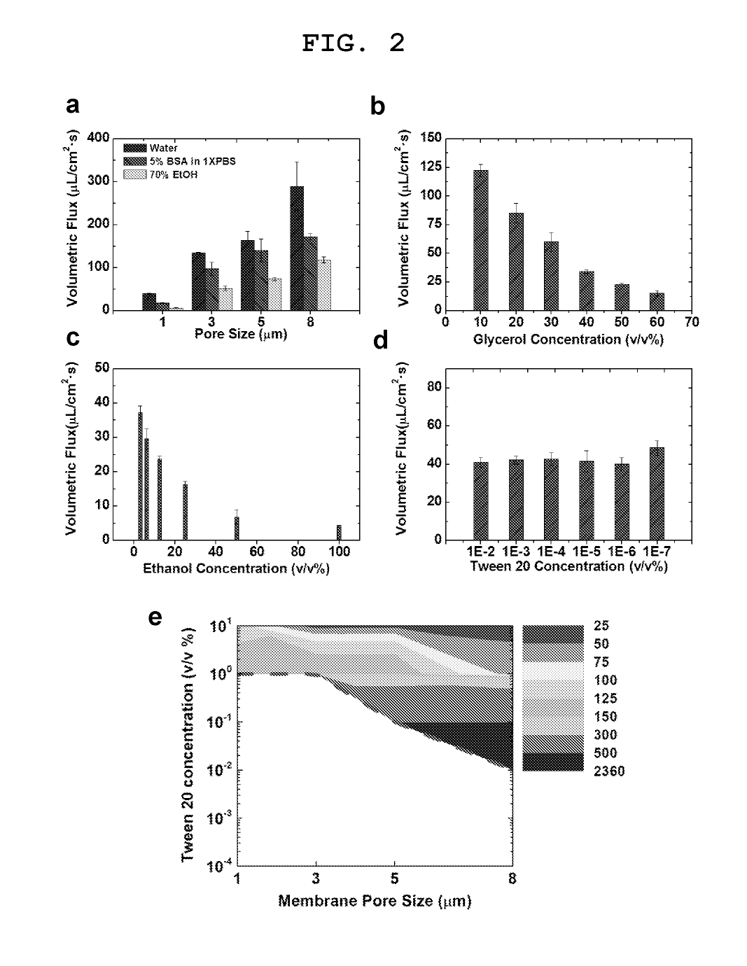

FIG. 3 shows mRNA isolation using the sieve-through platform. (a) Quantitative reverse transcriptase PCR analysis of mRNA isolated using the sieve-through platform as compared to the synthetic control. The two standard curves match well, indicating high isolation efficiency [sieve trough line is left low line]. (b) Quantitative reverse transcriptase PCR analysis of mRNA isolated from DLD-1 cells using the sieve-through platform and the conventional microcentrifuge tube [sieve trough line is lower line].

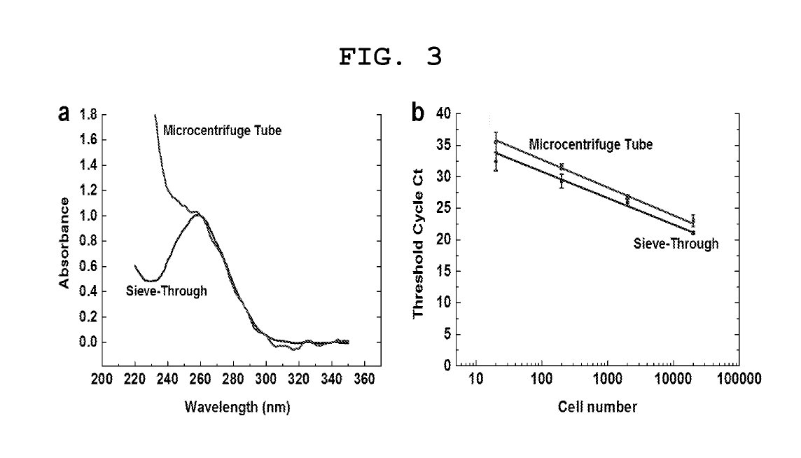

FIG. 4 shows isolating DNA with high-purity using the sieve-through platform. (a) normalized UV absorption spectra of isolated DNA. Abnormal tail at short wavelength was observed in DNA isolated using the conventional procedure in the microcentrifuge tube, which resulted in abnormal 260/230 ratio [sieve trough line is higher line]. (b) Real-time PCR analysis of isolated DNA. Smaller Ct value was observed for DNA isolated using the sieve-through platform, indicating high purity and/or high yield [sieve trough line is lower line].

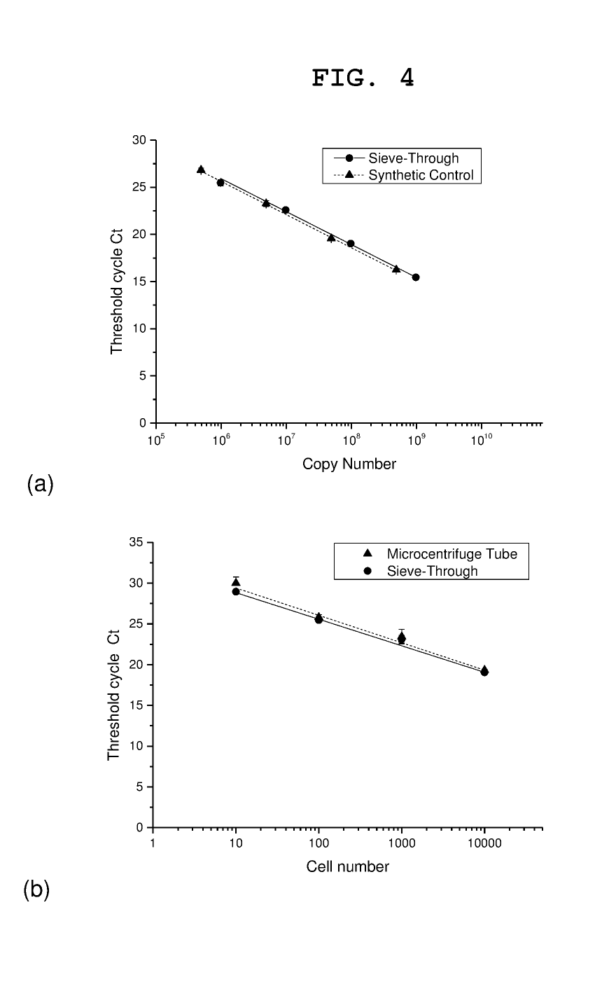

FIG. 5 shows a particle-based ELISA on the sieve-through platform. (a) Comparison between particle-based ELISA performed on the sieve-through platform and in the microcentrifuge tube. The sieve-through platform gives a lower background. (b) ELISA standard curve for AFP measured with 5-μm and 10-μm particles on the membrane with 3-μm pores. (c) The effect of particle antibody loading on the ELISA outcome. (d) The detection of AFP from whole blood.

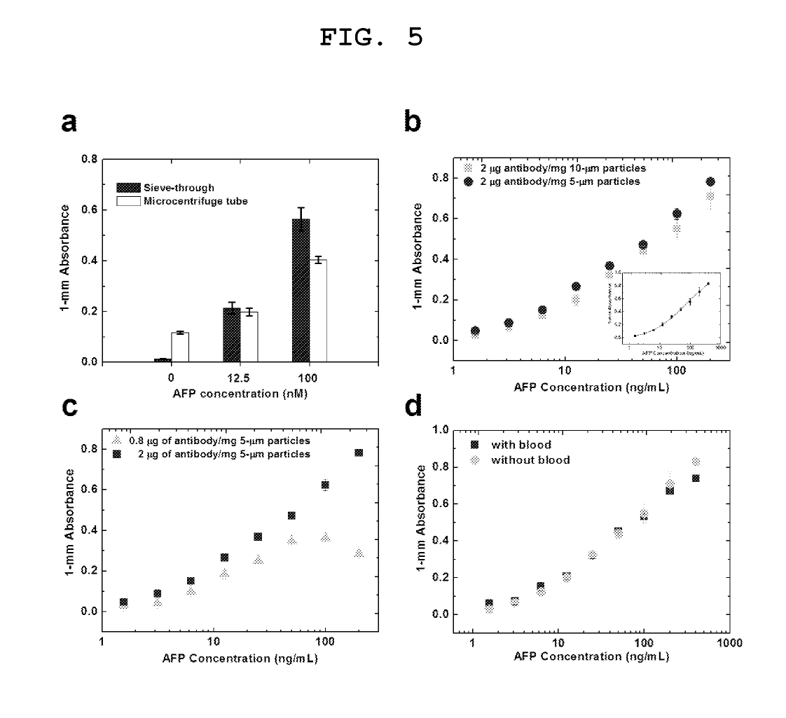

FIG. 6 shows a CD4+ cell count on the sieve-through platform device. (a) Direct quantification of CD₄₊ cells on the sieve-through platform. (b) The effect of the pore size on the cell count on the sieve-through platform. (c) The effect of tagging particles on the cell count on the sieve-through platform. (d) The false-colored SEM image of the CD4+ cells with tagging particles on the porous membrane. e) The SEM image showing overlapping pores. (f) CD₄₊ cell count from whole blood vs. 1×PBSB (1×PBS supplemented with 1% (w/v) BSA).

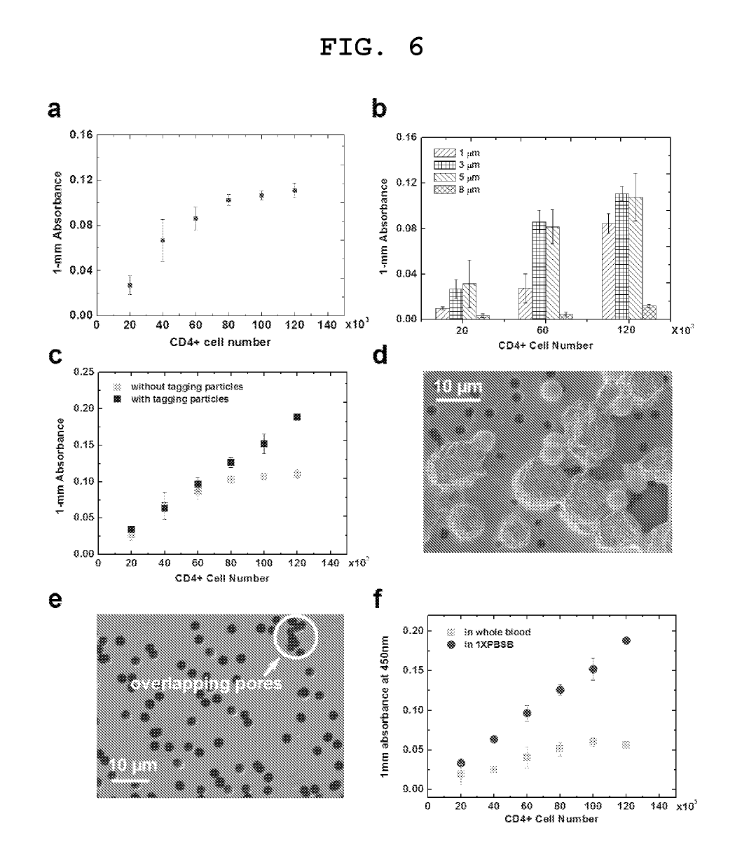

FIG. 7 shows photographs of the fabrication process of a high-throughput sieve-through vertical flow platform. (a) Membrane and 96-well plate alignment. (b) Heat application for the thermal bonding of membrane and plate, alignment is secured initially using tape (shown in green). (c) Bottom view of the device after thermal bonding.

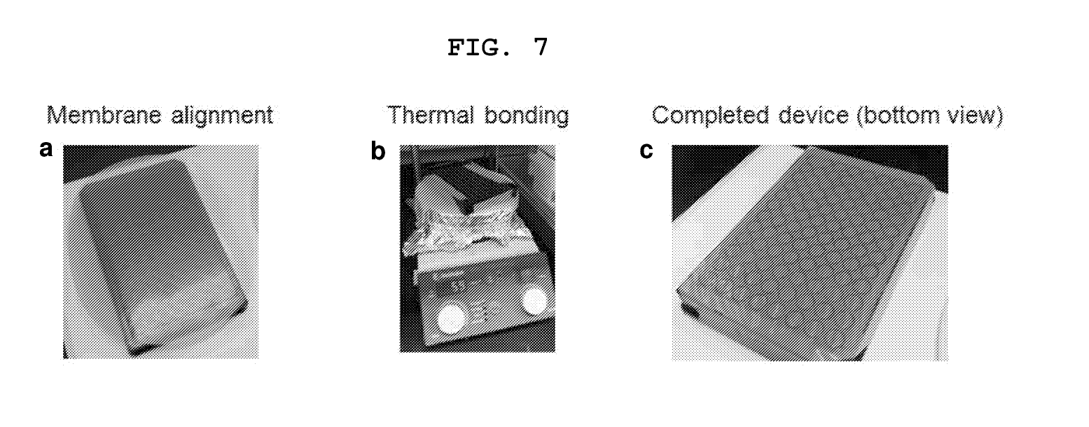

FIG. 8 shows that a significantly higher absorbance was obtained at 260 nm for mRNA extracted using the high-throughput sieve-through vertical flow platform (a), as compared to DynaBeads (b). (c) A comparable 260/280 absorbance ratio was achieved for the two approaches.

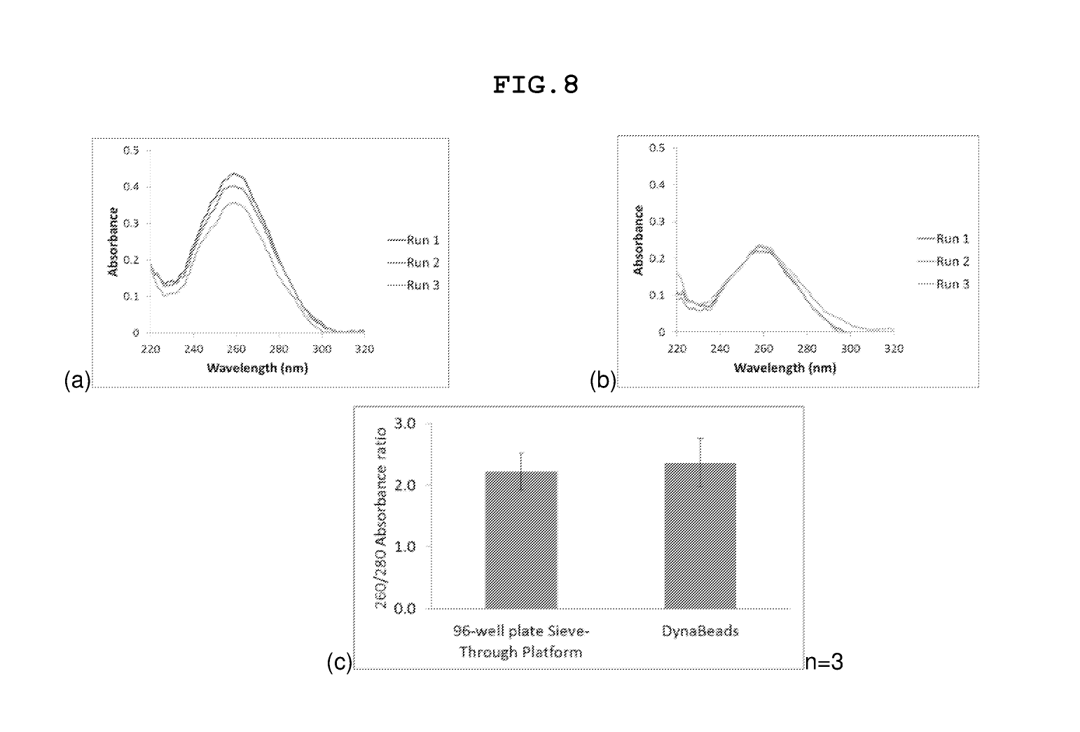

FIG. 9 shows that a significantly higher concentration of mRNA is extracted using the high-throughput sieve-through vertical flow platform, as compared to DynaBeads. (n=3).

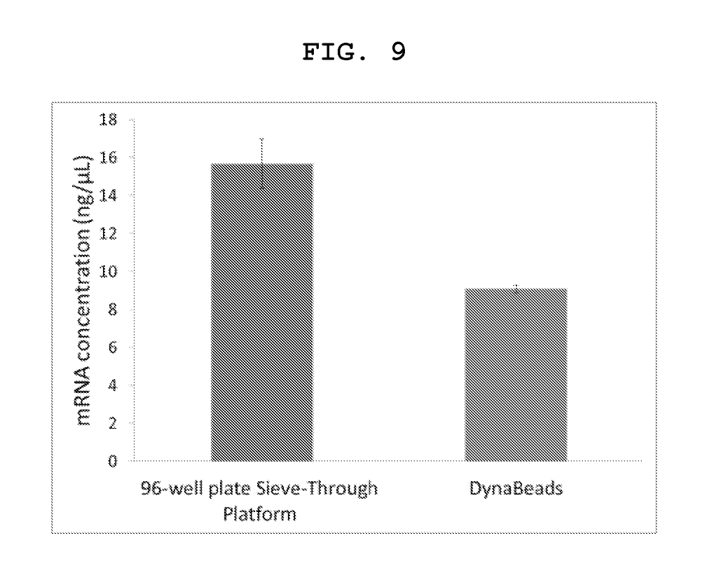

FIG. 10 shows that a lower elute volume is recovered by the high-throughput sieve-through vertical flow platform (via centrifugation), as compared to DynaBeads (via aspiration by pipette). (n=3).

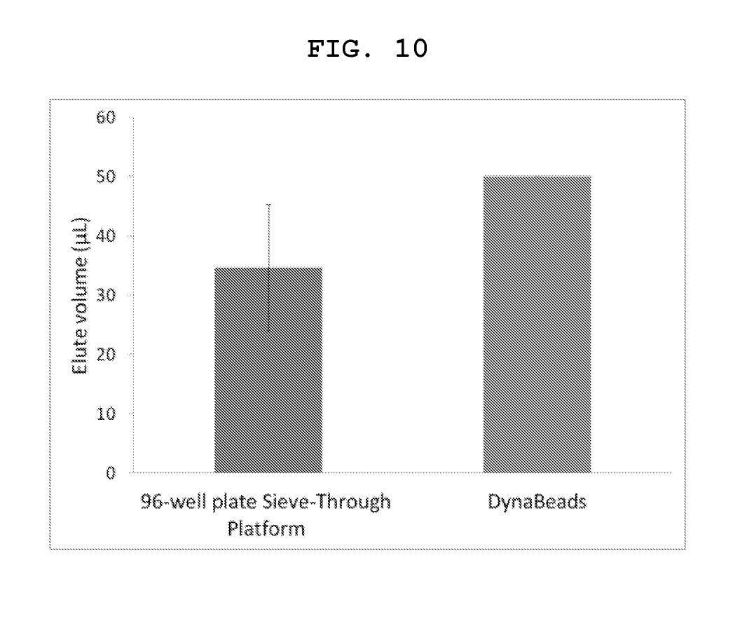

FIG. 11 shows that a higher absolute quantity of mRNA is extracted using the high-throughput sieve-through vertical flow platform, as compared to DynaBeads. (n=3).

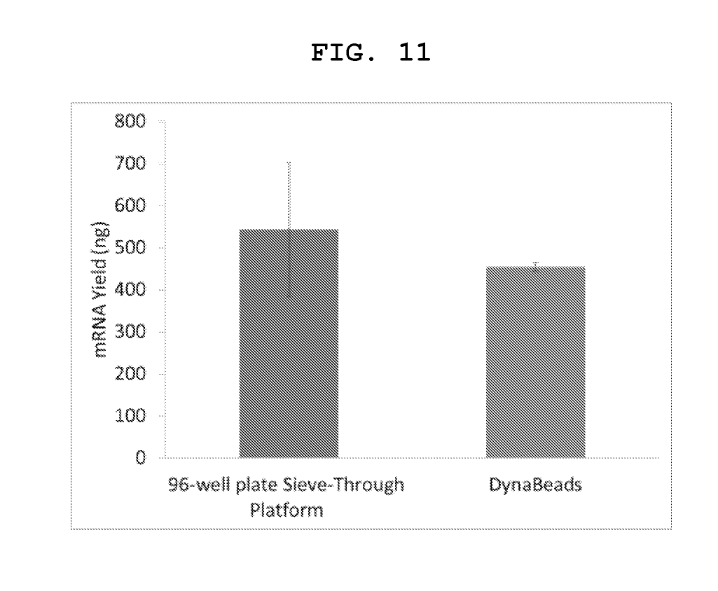

FIG. 12 shows a typical sieve-array: (a) Photograph of the sieve-array prototype; (b) Concurrent analysis of multiple samples on the sieve-array for potential high-throughput applications.

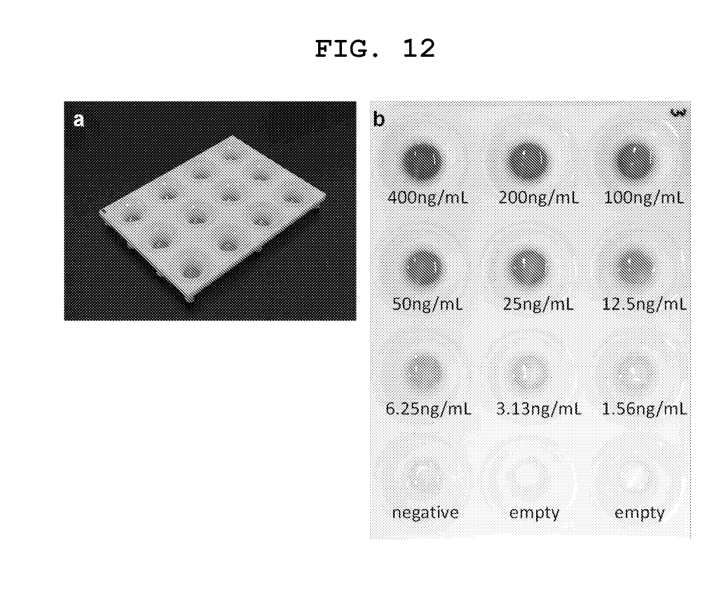

### INDUSTRIAL APPLICABILITY

The inventive device for liquid removal can have various applications in preparative and quantitative bioassays wherein a liquid is used and needs to be removed or replaced. The developed micro sieves can be utilized for sample preparation, immunoassays, ELISA etc. Such methods using the inventive concept are also part of the invention as described above.

The efficient removal of reaction solvents including contaminants therein is simple and can be used for high through-put applications, if arrays of the inventive device units are used.

The inventive device, arrays and methods may be used in automatable ELISA, as well as DNA and mRNA isolation assays which are commercially available as part of bioanalytical methods and research tools.

It will be apparent that various other modifications and adaptations of the invention are available to the person skilled in the art after reading the foregoing disclosure without departing from the spirit and scope of the invention and it is intended that all such modifications and adaptations come within the scope of the appended claims.

1.-29. (canceled)

30. A device for liquid removal in particle-based assay systems comprising
a) a chamber with at least one opening for adding particles, liquids and optionally other materials and with at least one other opening for removing liquids from the chamber;
b) a porous or sieve-like membrane, attached to the chamber covering the at least one opening for removing the liquid, to prevent the flowing of a liquid out of the chamber when not in contact with an absorbent pad;
wherein the membrane has pores or holes with a diameter that is smaller than the particle diameter of the particles used in the assay; and
c) a liquid absorbent pad that can be attached to and detached from the outer side of the membrane not facing the inner chamber.

31. The device according to claim 30 wherein a liquid can be removed from the chamber by vertical flow and the at least one opening for adding reagents is on top or at an upper section of the chamber and the at least one other opening for removing liquids from the chamber is at the bottom or at a lower section of the chamber or
wherein the flow of a liquid through the membrane is a passive flow after contact with the absorbent pad or
wherein the membrane becomes permeable for the liquids upon contact with the liquid absorbent pad.

32. The device according to claim 30 wherein the chamber is a reaction chamber for performing a chemical or biochemical reaction in a liquid.

33. The device according to claim 30 wherein the membrane is a microporous membrane made from a non-absorbent polymer.

34. The device according to claim 30 wherein the membrane prevents the liquid from flowing from the chamber with a hold-up time of more than 60 minutes or
wherein the membrane has pores or holes of a size of 0.05 to 50 μm.

35. The device according to claim 30 wherein the membrane is selected from polycarbonates, polyamides, modacrylic copolymers, styrene-acrylic acid copolymers, polysulfones, polyvinylidene fluoride, polyvinylfluoride, polychloroethers, thermoplastic polyethers, acetal polymers, polyacrylonitrile, polymethyl methacrylate, poly n-butyl methacrylate, polyurethanes, polyimides, polybenzimidazoles, polyvinyl acetate, aromatic and aliphatic polyethers, cellulose esters, epoxy resins, polyethylene, polypropylene, porous rubber, poly(ethylene oxides, polyvinylpyrrolidones, poly(vinyl alcohols), poly(sodium styrenesulfonate), polyvinylbenzyitrimethyl-ammonium chloride, poly(hydroxyethyl methacrylate), poly(isobutyl vinyl ether), polyisoprenes, polyalkenes, ethylene vinyl acetate copolymers, polyamides, and polyurethanes.

36. The device according to claim 30 wherein the absorbent pad is made from a hydrophilic material.

37. The device according to claim 30 wherein the particle based assay is preparative or analytical bioassay.

38. The device according to claim 30 wherein the particle is optionally functionalized and is selected from silica particles, polymeric particles, magnetic or superparamagnetic particles.

39. The device according to claim 30 wherein the particles are deposited on the membrane or on the chamber inner walls.

40. An array of devices according to claim 30 wherein the membranes of all or several devices can be contacted by a single absorbent pad or multiple sets of absorbent pads either simultaneously or at staggered sequence.

41. The array according to claim 40 wherein the array consist of a well plate wherein the membrane is attached to the bottom of the well plate by adhesive, double-sided tape, polydimethylsiloxane (PDMS) or thermal bonding of the membrane to the plate bottom or wherein the membrane is a polycarbonate membrane with micrometer-size holes.

42. A preparative or quantitative assay method, comprising:
a) providing a chamber with at least one opening for adding particles, liquids and optionally other materials;
and with at least one other opening for removing liquids from the chamber which is covered by a porous or sieve-like membrane;
b) filling particles and at least one liquid into the chamber together with a preparation of a sample material and optional reagents and/or other materials into the chamber;
c) performing a reaction in the chamber wherein the particles react or interact with a preparation of a sample material without any substantial flow of liquid through the membrane;
d) removing the reaction liquids and optionally dissolved by-products via the porous or sieve-like membrane by causing a flow through the membrane after contacting the membrane with an absorbent pad at the outer side of the membrane not facing the inner chamber;
e) optionally adding a liquid to the chamber; and
f) detecting or collecting the sample material that has reacted with the particle.

43. The method according to claim 42 wherein the chamber is filled with a washing liquid after operation d) and thereafter this washing liquid is removed through the membrane by further contacting the membrane the absorbent pad before performing to operation e) or f).

44. The method according to claim 42 wherein the particles are polymeric particles functionalized with a labelled capture antibody and the preparation of the sample material comprises a enzyme-labeled detector antibody or wherein the particles interact with the sample material to form sample materials which are tagged by the particle by adherence or chemical binding.

45. The method according to claim 42 wherein the preparation comprises DNA and the particle is a magnetic particle or wherein the preparation comprises mRNA and the particle is a poly(T) conjugated magnetic particle.

46. The method according to claim 42 wherein the particles are first filled into the chamber in operation b) together with a liquid, the liquid is removed and the particle deposited on the membrane and or/chamber walls before the sample material together with at least one liquid is filled in the chamber, or wherein the sample material is tagged to a particle during operation c).

47. A preparative or quantitative assay method, comprising:
a) providing a chamber with at least one opening for adding particles, liquids and optionally other materials
and with at least one other opening for removing liquids from the chamber which is covered by a porous or sieve-like membrane;
b) filling sample material in particle form or immobilized on particles in a liquid preparation and optional reagents or fillers into the chamber;
c) removing the liquids and optionally dissolved substances via the porous or sieve-like membrane by causing a flow through the membrane after contacting the membrane with an absorbent pad at the outer side of the membrane not facing the inner chamber;
d) optionally adding a liquid to the chamber; and
e) detecting or collecting the sample material in particle form or immobilized on the particles.

48. The method according to claim 47 wherein the chamber is filled with a washing liquid after operation d) and thereafter this washing liquid is removed through the membrane by further contacting the membrane the absorbent pad before performing to operation d) or e).

49. The method according to claim 47 wherein the sample material immobilized on particles comprises DNA or RNA and the particles are selected from silica beads or silica-coated magnetic beads or
wherein the sample material immobilized on particles comprises mRNA and the particles are selected from oligo(dT) magnetic beads.
